(19) United States
(12) Patent Application Publication
(10) Pub. No.: US 2026/0354707 A1
(43) Pub. Date: Nov. 12, 2026

(54) Movement Quality Classification Using Multi-Axis Motion Sensing

(71) Applicant: Amy Jo Lambert, East Peoria, IL (US)

(72) Inventor: Amy Jo Lambert, East Peoria, IL (US)

(21) Appl. No.: 19/669,957

(22) Filed: May 6, 2026

Related U.S. Application Data
(60) Provisional application No. 63/803,771, filed on May 11, 2025.

(51) Int. Cl. A61B 5/11; G01P 15/18

(52) U.S. Cl. CPC A61B 5/1126; G01P 15/18

(57) ABSTRACT

A system and method for classifying movement quality using motion data are disclosed. A sensing system captures motion data of a user. A processing system filters the motion data, segments the motion data into time-based windows and extracts motion features, including acceleration, rotational characteristics, periodicity and relationships among motion features. An interpretation module classifies movement quality based on relationships among the extracted motion features rather than predefined exercise templates, activity-specific models or isolated feature values. The movement quality classification may include stability, smoothness, control, coordination, coherence or variability and may be expressed as a numerical score, categorical classification or structured movement-quality state representation. An output system generates responsive feedback based on the classification. The system may support wearable, non-wearable or hybrid sensing configurations, placement-aware interpretation, user-initiated sensing, continuous sensing, intermittent sensing and tracking of movement quality over time.

### CROSS-REFERENCE TO RELATED APPLICATIONS

This application claims the benefit of U.S. Provisional Patent Application No. 63/803,771, filed May 11, 2025, entitled Poemme: Gracefulness Measurement and Emotionally-Responsive Narrative System, the entire contents of which are incorporated herein by reference.

### FIELD OF THE INVENTION

The present invention relates generally to motion sensing and movement analysis systems and, more particularly, to systems and methods for classifying movement quality using multi-axis motion sensing.

### BACKGROUND OF THE INVENTION

Conventional wearable motion sensing systems and fitness trackers primarily measure quantitative activity metrics such as step count, heart rate, calories, distance, intensity or duration of movement. While such metrics may provide useful information regarding activity level, they generally do not evaluate qualitative aspects of movement, such as stability, smoothness, control, coordination, coherence, rhythmic consistency or variability.

Existing systems may classify particular exercise types, detect predefined gestures or measure isolated motion parameters. However, such systems often rely on predefined activity templates, exercise-specific models or motion patterns associated with known gestures. These approaches are limited because human movement quality may vary across arbitrary movement patterns that do not correspond to a predefined exercise, gesture or activity class.

There is therefore a need for a system capable of capturing motion data, extracting relationships among motion features, classifying movement quality across arbitrary movement patterns and providing responsive feedback based on qualitative movement characteristics. There is also a need for such a system to operate across different sensing configurations, including wearable, non-wearable and hybrid sensing systems and to adjust interpretation based on sensor placement, session context or changes in movement over time.

### SUMMARY OF THE INVENTION

The present invention provides a system and method for capturing, processing and classifying human movement quality using multi-axis motion sensing.

In one embodiment, the system includes a sensing system configured to capture motion data of a user. The sensing system may include one or more wearable sensing modules, non-wearable sensing systems or a combination thereof. A wearable sensing module may include a multi-axis inertial measurement unit having at least an accelerometer and a gyroscope. Non-wearable sensing systems may include camera-based systems, depth sensors, environmental sensors or other non-contact sensing technologies.

The system further includes a processing system configured to receive motion data, filter the motion data, segment the motion data into time-based windows and extract motion features. Motion features may include acceleration magnitude, rotational velocity, angular displacement, velocity, periodicity, jerk, signal variability, temporal alignment, amplitude correlation, frequency synchronization or relationships among such features.

An interpretation module classifies movement quality based on relationships among extracted motion features. The classification may be performed independent of predefined motion templates, predefined exercise classifications or activity-specific models and may be applicable across arbitrary movement patterns. Movement quality may include stability, smoothness, control, coordination, coherence, rhythmic consistency or variability. The classification may be expressed as a numerical movement-quality score, a categorical classification, a structured movement-quality state representation or a combination thereof.

In some embodiments, the interpretation module evaluates relationships among motion features across sequential time windows to determine consistency, variability or changes in movement quality over time. The system may classify movement quality at a segment level, session level, longitudinal level or combinations thereof.

In some embodiments, the interpretation module adjusts classification based on placement of the sensing system relative to the user's body. Placement may be defined by user input, inferred from motion patterns or determined based on sensor configuration.

The system includes an output system configured to generate responsive feedback based on the movement quality classification. Responsive feedback may include haptic feedback, visual feedback, audio feedback, informational output or environmental output. Responsive feedback may be provided through a wearable sensing module, connected computing device, mobile application, display interface or other output device.

The system may support user-initiated sensing sessions, automatically initiated sensing sessions, continuous sensing, intermittent sensing and termination of sensing based on user inactivity or expiration of a predefined time interval.

### BRIEF DESCRIPTION OF THE DRAWINGS

FIG. 1 is a block diagram of a system for classifying movement quality using a sensing system, processing system, interpretation module and output system.

FIG. 2 is a flow diagram illustrating a method for capturing motion data, filtering and segmenting the motion data, extracting motion features, classifying movement quality and generating responsive feedback.

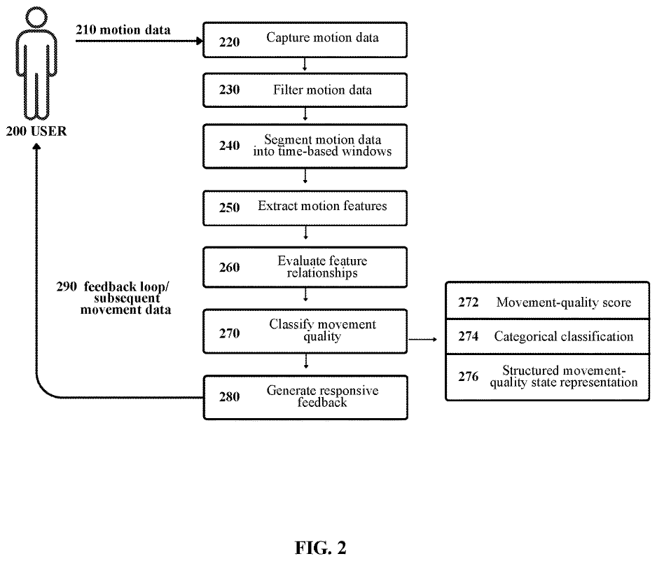

FIG. 3 is a diagram illustrating movement-quality classification across sequential time windows and placement-aware interpretation of motion data from different body locations or sensing configurations.

### DETAILED DESCRIPTION

The present invention provides a system and method for capturing, processing and classifying human movement quality using multi-axis motion sensing. The system may be implemented using wearable sensing devices, non-wearable sensing devices, connected computing devices, cloud-based processing systems, local processing systems or combinations thereof.

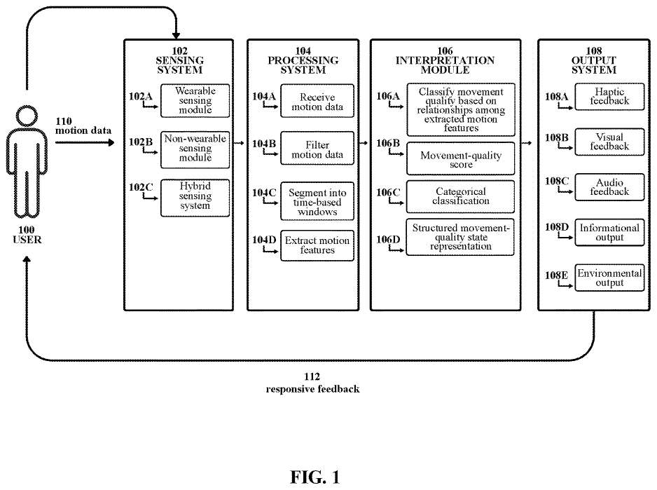

Referring to FIG. 1, a system for classifying movement quality may include a user 100, a sensing system 102, a processing system 104, an interpretation module 106 and an output system 108. Motion data 110 associated with movement of the user 100 is provided to the sensing system 102. Responsive feedback 112 may be provided from the output system 108 to the user 100.

### Sensing System

In one embodiment, the sensing system 102 is configured to capture motion data 110 associated with movement of the user 100. The sensing system 102 may include one or more wearable sensing modules 102A configured to be worn on or attached to the user's body. The wearable sensing module 102A may include a multi-axis inertial measurement unit comprising at least an accelerometer and a gyroscope. In some embodiments, the wearable sensing module 102A may further include a magnetometer, barometer, biometric sensor, haptic output mechanism, wireless communication interface, processor, memory or power source.

The wearable sensing module 102A may be configured for head-worn use, wrist-worn use, chest-mounted use, arm-worn use, ankle-worn use, waist-mounted use, garment-integrated use, jewelry-integrated use or other body placement configurations.

In some embodiments, the sensing system 102 may include a non-wearable sensing system 102B. The non-wearable sensing system 102B may include camera-based systems, depth sensors, optical sensors, radar sensors, lidar sensors, pressure sensors, environmental sensors or other non-contact sensing technologies configured to capture motion data 110 of the user 100.

In still other embodiments, motion data 110 may be captured using a hybrid sensing system 102C including a combination of wearable and non-wearable sensing systems. Accordingly, motion data 110 may be obtained through contact-based sensing, non-contact sensing or hybrid sensing configurations.

### Session Initiation and Sensing Modes

The sensing system may include a user input mechanism configured to initiate a motion capture session. The user input mechanism may include a button, touch interface, gesture input, mobile application control, voice command or other user interface.

In some embodiments, a motion capture session may be initiated automatically based on detection of motion, detection of a user state, scheduled timing, proximity to a device, environmental context or a command from another system. The system may also support continuous sensing as an operational mode.

In some embodiments, the system performs intermittent motion data sampling outside of active motion capture sessions. Intermittent sampling may be used to detect changes in movement quality, conserve power, identify transitions between active and inactive states or trigger a more detailed motion capture session.

A motion capture session may terminate based on detection of user inactivity, expiration of a predefined time interval, user input, battery state, completion of a movement sequence, completion of a feedback cycle or satisfaction of another termination condition.

### Processing System

Referring again to FIG. 1, the processing system 104 may be configured to perform a receiving operation 104A, a filtering operation 104B, a segmentation operation 104C and a feature-extraction operation 104D.

Motion data captured by the sensing system is provided to a processing system. The processing system may be located within the wearable sensing module, within a connected computing device, within a cloud computing environment or distributed across multiple devices.

The processing system is configured to receive motion data and perform signal processing operations. Signal processing operations may include filtering the motion data to reduce noise, removing artifacts, normalizing signal values, correcting drift, synchronizing multiple sensors, aligning sampling windows or compensating for sensor placement.

The processing system segments the motion data into time-based windows. The time-based windows may be overlapping or non-overlapping. Segment duration may be fixed, variable, user-specific, activity-specific or dynamically adjusted based on motion characteristics.

The processing system extracts one or more motion features from the segmented motion data. Motion features may include acceleration magnitude, rotational velocity, angular velocity, angular displacement, orientation change, velocity, periodicity, signal variability, jerk, sway, cadence, smoothness, symmetry, rhythm, transition timing or other motion-derived values.

In some embodiments, the processing system extracts relationship features among motion features. Relationship features may include temporal alignment, amplitude correlation, frequency synchronization, phase relationships, cross-axis coordination, inter-axis variability, rhythm consistency or other interdependencies among extracted motion features.

### Interpretation Module

Referring again to FIG. 1, the interpretation module 106 may include classification logic 106A configured to classify movement quality based on relationships among extracted motion features. The interpretation module 106 may generate a movement-quality score 106B, a categorical classification 106C, a structured movement-quality state representation 106D or combinations thereof.

An interpretation module is configured to classify movement quality based on the extracted motion features. The interpretation module may be executed by the processing system, by a connected computing device, by a cloud-based system or by a distributed computing architecture.

The interpretation module classifies movement quality based on relationships among extracted motion features rather than based only on isolated feature values. In some embodiments, the interpretation module evaluates interdependencies or interactions among the motion features whether explicitly computed or implicitly learned by a model.

Movement quality may include stability, smoothness, control, coordination, coherence, rhythmic consistency, variability, fluidity or other qualitative characteristics of movement.

In some embodiments, the classification is performed independent of predefined motion templates, predefined exercise classifications or activity-specific models. The classification may therefore be applicable across arbitrary movement patterns, including movements that are not associated with a predefined exercise, gesture, activity or rehabilitation protocol.

The interpretation module may generate a numerical movement-quality score, a categorical movement-quality classification or both. Numerical scores may be normalized, scaled, weighted or combined across multiple motion features or relationship features. Categorical classifications may include classifications such as stable, unstable, smooth, irregular, controlled, uncontrolled, coordinated, uncoordinated, improving, declining or other descriptive classifications.

In some embodiments, movement quality may be represented as a structured movement-quality state representation. The structured movement-quality state representation may include a vector, matrix, classification object, latent representation, model output or other data structure representing qualitative movement characteristics.

### Sequential Time Window Evaluation

The interpretation module may evaluate relationships among extracted motion features over sequential time windows. Such evaluation may determine consistency, variability, change, improvement, decline or trajectory of movement quality over time.

Movement quality may be evaluated at a segment level, session level, longitudinal level or combinations thereof. Segment-level evaluation may classify movement quality within a particular time window. Session-level evaluation may aggregate or summarize movement quality across a motion capture session. Longitudinal evaluation may compare movement quality across multiple sessions or time periods.

In some embodiments, the system tracks changes in movement quality over time. Tracking may include storing movement-quality scores, categorical classifications, state representations, trend data or session summaries. The stored data may be used to identify changes in stability, control, coordination or other qualitative movement characteristics.

### Placement-Aware Interpretation

In some embodiments, the interpretation module is configured to adjust movement-quality classification based on placement of the sensing system relative to the user's body. Placement may include head placement, wrist-worn placement, chest-mounted placement, waist-mounted placement, ankle placement, garment placement, jewelry placement or other body locations.

Placement may be defined by user input, selected through a connected computing device or inferred based on motion patterns. In some embodiments, placement may be inferred by comparing detected motion patterns to expected sensor-placement signatures.

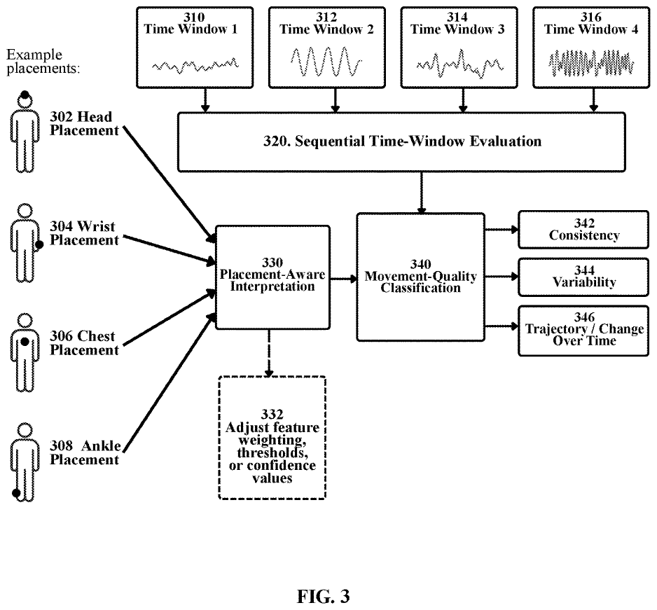

Referring to FIG. 3, placement-aware interpretation may account for different sensing locations, including head placement, wrist placement, chest placement and ankle placement.

Placement-aware interpretation may modify feature weighting, thresholds, signal normalization, classification logic, expected ranges or confidence values. For example, wrist-worn motion data may emphasize arm movement patterns while chest-mounted motion data may emphasize torso stability, postural control or whole-body coordination.

### Output System

The output system 108 may generate responsive feedback 112 based on the movement-quality classification. Responsive feedback 112 may include haptic feedback 108A, visual feedback 108B, audio feedback 108C, informational output 108D, environmental output 108E or combinations thereof.

Responsive feedback may be provided by a wearable sensing module, connected computing device, mobile application, display interface or other output device.

Haptic feedback may include vibration patterns, pulses, intensity changes or timing patterns. Haptic feedback may communicate system state transitions including activation, deactivation, sensing status, classification status, movement-quality alerts or session completion.

Informational output may include numerical scores, categorical classifications, trend indicators, session summaries, prompts, alerts, coaching messages or visualizations. Informational output may be presented through a mobile application, desktop application, web interface, wearable display, external display or other computing interface.

In some embodiments, responsive feedback may be generated based on a current movement-quality classification, a change in movement quality, a movement-quality threshold, a trend over time or comparison with prior movement data.

### Feedback and Adaptation

The system may operate in a feedback loop in which movement data is captured, movement quality is classified, responsive feedback is generated and subsequent movement data is captured to evaluate changes after feedback.

In some embodiments, responsive feedback may be adapted based on user response, movement-quality trajectory, sensor confidence, placement, user preference or session context. For example, feedback intensity may be increased when movement instability is detected or reduced when movement quality improves.

The system may also adjust sampling frequency, sensing mode, classification thresholds or feedback modality based on detected movement quality or user response.

### Computing Environment

The functions described herein may be performed by one or more processors executing instructions stored in memory. The processing system may include local processors, wearable processors, mobile processors, cloud processors, edge processors or distributed processing systems.

The system may include one or more wireless communication interfaces. Wireless communication interfaces may include Bluetooth, Bluetooth Low Energy, Wi-Fi, cellular, near-field communication or other communication protocols.

Data may be stored locally, remotely or in a distributed manner. Stored data may include raw motion data, processed motion data, extracted features, classification outputs, session summaries, sensor placement data, user preferences or longitudinal trend data.

What is claimed is:

1. A system for classifying movement quality, comprising:
a sensing system configured to capture motion data of a user;
a processing system configured to receive the motion data, filter the motion data, segment the motion data into time-based windows and extract one or more motion features from the motion data;
an interpretation module configured to classify movement quality of the user based on relationships among the extracted motion features; and
an output system configured to generate responsive feedback based on the movement quality classification;
wherein the movement quality classification is performed independent of predefined motion templates, predefined exercise classifications or activity-specific models and is applicable across arbitrary movement patterns.

2. The system of claim 1, wherein the sensing system comprises a wearable sensing module including a multi-axis inertial measurement unit.

3. The system of claim 2, wherein the multi-axis inertial measurement unit includes at least an accelerometer and a gyroscope.

4. The system of claim 1, wherein the sensing system comprises at least one of a wearable sensing module, a non-wearable sensing system or a hybrid sensing system.

5. The system of claim 1, wherein the one or more motion features include at least one of acceleration magnitude, rotational velocity, angular velocity, velocity, periodicity, jerk, signal variability or movement rhythm.

6. The system of claim 1, wherein the relationships among the extracted motion features include at least one of temporal alignment, amplitude correlation, frequency synchronization, phase relationship, cross-axis coordination or inter-axis variability.

7. The system of claim 1, wherein the movement quality classification includes at least one of stability, smoothness, control, coordination, coherence, rhythmic consistency or variability.

8. The system of claim 1, wherein the interpretation module is configured to generate at least one of a numerical movement-quality score, a categorical movement-quality classification or a structured movement-quality state representation.

9. The system of claim 8, wherein the structured movement-quality state representation includes at least one of a vector, matrix, classification object, latent representation or model output.

10. The system of claim 1, wherein the interpretation module is configured to classify movement quality based on relationships among the extracted motion features across sequential time windows.

11. The system of claim 1, wherein the interpretation module is configured to classify movement quality at both a segment level and a session level.

12. The system of claim 1, wherein the interpretation module is configured to adjust movement quality classification based on placement of the sensing system relative to the user's body.

13. The system of claim 12, wherein the placement is defined by user input or inferred based on motion patterns.

14. The system of claim 1, wherein the output system generates at least one of haptic feedback, visual feedback, audio feedback, informational output or environmental output.

15. The system of claim 1, wherein the processing system is configured to track changes in movement quality over time.

16. The system of claim 1, wherein the output system adapts responsive feedback based on a change in movement quality over time.

17. The system of claim 1, wherein the system supports at least one of user-initiated sensing, automatically initiated sensing, continuous sensing or intermittent sensing.

18. A method for classifying movement quality, comprising:
capturing motion data of a user using a sensing system;
filtering the motion data;
segmenting the motion data into time-based windows;
extracting one or more motion features from the motion data;
classifying movement quality of the user based on relationships among the extracted motion features; and
generating responsive feedback based on the movement quality classification;
wherein the movement quality classification is performed independent of predefined motion templates, predefined exercise classifications or activity-specific models and is applicable across arbitrary movement patterns.

19. The method of claim 18, further comprising at least one of generating a numerical movement-quality score, generating a categorical movement-quality classification, generating a structured movement-quality state representation, adjusting classification based on sensor placement or tracking changes in movement quality over time.

20. A non-transitory computer-readable medium storing instructions that, when executed by one or more processors, cause the one or more processors to perform operations comprising:
receiving motion data associated with movement of a user;
filtering the motion data;
segmenting the motion data into time-based windows;
extracting one or more motion features from the motion data;
classifying movement quality of the user based on relationships among the extracted motion features; and
generating responsive feedback based on the movement quality classification;
wherein the movement quality classification is performed independent of predefined motion templates, predefined exercise classifications or activity-specific models and is applicable across arbitrary movement patterns.
